(19) United States
(12) Patent Application Publication
(10) Pub. No.: US 2026/0185035 A1
(43) Pub. Date: Jul. 2, 2026

(54) METHODS, DEVICES, AND APPARATUS FOR SEPARATING CELLS BY TILTING

(71) Applicant: CURIOX BIOSYSTEMS CO., LTD., Seoul (KR)

(72) Inventors: Kok Siang TAN, Seoul (KR); Chyan Ying KE, Seoul (KR); Namyong KIM, Singapore (SG)

(21) Appl. No.: 19/547,525

(22) Filed: Feb. 23, 2026

Related U.S. Application Data
(63) Continuation of application No. PCT/IB2024/000436, filed on Aug. 21, 2024.
(60) Provisional application No. 63/520,923, filed on Aug. 21, 2023.

(51) Int. Cl. C12M 1/00; C12M 3/06

(52) U.S. Cl. CPC C12M 47/04; C12M 27/16

(57) ABSTRACT

A method includes maintaining for a first period of time a receptacle defining a first channel in a first orientation so that a first axis defined by the first channel is in a non-vertical direction, and maintaining for a second period of time subsequent to the first period of time the receptacle in a second orientation that is distinct from the first orientation. In some cases, the receptacle includes a solution containing biological cells in the first channel.

### RELATED APPLICATION

This application is a continuation application of International Patent Application No. PCT/IB2024/000436, filed Aug. 21, 2024, which the claims the benefit of, and priority to, U.S. Provisional Patent Application Ser. No. 63/520,923, filed Aug. 21, 2023. Each of these applications is incorporated by reference herein in its entirety.

### TECHNICAL FIELD

This application relates to methods, devices, and apparatus for separating biological cells or other particles.

### BACKGROUND

Separation of biological cells is a critical step in many biological processes and assays. Conventional methods for separating biological cells include subjecting the biological cells in a liquid to a large acceleration, such as by using a centrifuge. The centrifugal force causes the biological cells to travel to the bottom of a tube and form a precipitate, allowing a remaining liquid to be separated from the precipitate.

However, the mechanical force applied on the biological cells as well as formation of the precipitate may affect the biological cells. For example, too much centrifugal force will cause lysis of biological cells.

### SUMMARY

Accordingly, there is need for methods, devices, and apparatus that do not require large acceleration for separating biological cells. Such methods, devices, and apparatus plates may replace the conventional methods, devices, and apparatus for separating biological cells. Such methods, devices, and apparatus may better retain physical and biological properties of cells during separation by reducing or eliminating the need for applying large acceleration or force, thereby enhancing the accuracy or efficiency of biological processes and assays. Such methods, devices, and apparatus may also be used in washing other types of samples, such as beads or particles conjugated with biological molecules.

A number of embodiments that overcome the limitations and disadvantages of existing methods, devices, and apparatus are presented in more detail below. These embodiments provide methods, devices, and apparatus for separating biological cells.

As described in more detail below, in accordance with some embodiments, a method includes maintaining for a first period of time a receptacle defining a first channel in a first orientation so that a first axis defined by the first channel is in a non-vertical direction. The receptacle includes a solution containing biological cells in the first channel. The method also includes maintaining for a second period of time subsequent to the first period of time the receptacle in a second orientation that is distinct from the first orientation.

In accordance with some embodiments, an apparatus includes a receptacle holder for holding a receptacle, and a tilting device coupled with the receptacle holder for placing the receptacle holder in a first orientation at a first time and placing the receptacle holder in a second orientation distinct from the first orientation at a second time distinct from the first time.

In accordance with some embodiments, a method for separating non-cellular substances from biological cells in a solution includes maintaining for a first period of time a receptacle defining a first channel in a first orientation so that a first axis defined by the first channel is in a non-vertical direction. The receptacle includes the solution in the first channel, the solution containing the biological cells and the non-cellular substances. The method also includes maintaining for a second period of time subsequent to the first period of time the receptacle in a second orientation that is distinct from the first orientation; and aspirating a portion, less than all, of the solution.

In accordance with some embodiments, a method includes maintaining for a first period of time a receptacle defining a first channel in a first orientation so that a first axis defined by the first channel is in a first non-vertical direction, the receptacle including a solution containing biological cells in the first channel; and maintaining for a second period of time subsequent to the first period of time the receptacle in a second orientation distinct from the first orientation so that the first axis defined by the first channel is in a second non-vertical direction distinct from the first non-vertical direction.

### BRIEF DESCRIPTION OF THE DRAWINGS

For a better understanding of the aforementioned embodiments as well as additional embodiments, reference should be made to the Description of Embodiments below, in conjunction with the following drawings in which like reference numerals refer to corresponding parts throughout the figures.

FIG. 1 is a perspective view of a plate in accordance with some embodiments.

FIG. 2 is a cross-sectional view of the plate shown in FIG. 1.

FIG. 3 illustrates a tube in accordance with some embodiments.

FIGS. 4A-4I illustrate devices for separating biological cells and their operations in accordance with some embodiments.

FIGS. 5 and 6 illustrate experimental results obtained by using the methods described herein.

FIGS. 7A-7C are flow diagrams illustrating a method of concentrating biological cells in accordance with some embodiments.

FIG. 8 is a flow diagram illustrating a method of separating non-cellular substances from biological cells in accordance with some embodiments.

FIG. 9 is a flow diagram illustrating a method of concentrating biological cells in a receptable defining a channel in accordance with some embodiments.

FIG. 10 is a block diagram illustrating electrical components of an apparatus in accordance with some embodiments.

FIGS. 11A and 11B are schematic diagrams illustrating impulse devices in accordance with some embodiments.

Like reference numerals refer to corresponding parts throughout the drawings.

Drawings are not necessarily drawn to scale unless indicated otherwise.

### DESCRIPTION OF EMBODIMENTS

Methods, devices, and apparatus for washing samples are described. Reference will be made to certain embodiments, examples of which are illustrated in the accompanying drawings. While the claims will be described in conjunction with the embodiments, it will be understood that it is not intended to limit the claims to these particular embodiments alone. On the contrary, the embodiments are intended to cover alternatives, modifications and equivalents that are within the spirit and scope of the appended claims.

Moreover, in the following description, numerous specific details are set forth to provide a thorough understanding of the embodiments. However, it will be apparent to one of ordinary skill in the art that the embodiments may be practiced without these particular details. In other instances, methods, procedures, components, and networks that are well-known to those of ordinary skill in the art are not described in detail to avoid obscuring aspects of the embodiments.

It will also be understood that, although the terms first, second, etc. may be used herein to describe various elements, these elements should not be limited by these terms. These terms are only used to distinguish one element from another. For example, a first channel could be termed a second channel, and, similarly, a second channel could be termed a first channel, without departing from the scope of the embodiments. The first channel and the second channel are both channels, but they are not the same channel. Similarly, a first period of time could be termed a second period of time, and, similarly, a second period of time could be termed a first period of time, without departing from the scope of the embodiments. The first period of time and the second period of time are both periods of time, but they are not the same period of time.

The terminology used in the description of the embodiments herein is for the purpose of describing particular embodiments only and is not intended to be limiting of the invention. As used in the description of the embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

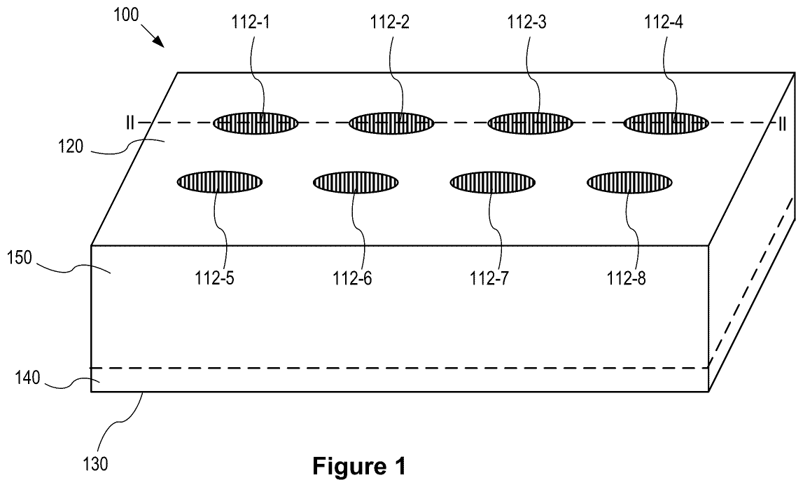

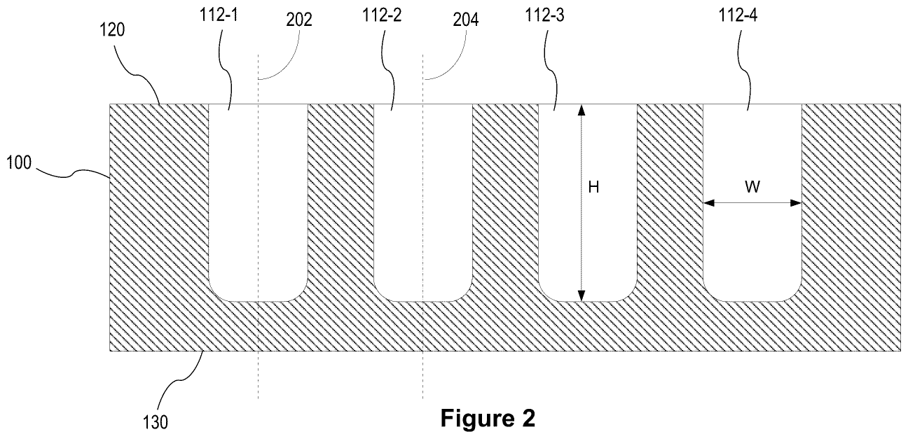

FIG. 1 is a perspective view of a receptacle (e.g., plate 100) in accordance with some embodiments. The plate 100 has a top surface 120 and a bottom surface 130 opposite to the top surface 120. A plurality of wells 112 (e.g., wells 112-1 through 112-8) is defined in the plate 100. The plate 100 includes a first portion 140 that corresponds to a bottom of the plurality of wells 112 and a second portion 150 that corresponds to one or more walls of the plurality of wells 112. In some embodiments, the plate 100 is formed integrally. In some embodiments, the plate 100 is formed by attaching two or more portions together (e.g., by bonding separately formed first and second portions 140 and 150). In some embodiments, as shown in FIG. 1, the plurality of wells 112 is arranged in an array (e.g., 2-by-3 array, 2-by-4 array, 3-by-4 array, 4-by-6 array, 6-by-8 array, 8-by-12 array, 16-by-24 array, 32-by-48 array, etc.). In some embodiments, a respective well 112 is a cylindrical well (e.g., a cross-section of the respective well 112 along a plane substantially parallel to the plate 100 has a shape of a circle). Also shown in FIG. 1 is line II-II, from which the cross-sectional view of FIG. 2 is taken.

FIG. 2 is a cross-sectional view of the plate 100 shown in FIG. 1. In some embodiments, a respective well 112 has a width (W) of 2 mm- 170 mm (e.g., 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 160 mm, 170 mm, or any interval between two of the aforementioned values, such as 5 mm- 8 mm). In some embodiments, the respective well 112 has a height (H) of 2 mm- 170 mm (e.g., 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 160 mm, 170 mm, or any interval between two of the aforementioned values, such as 3 mm- 70 mm).

FIG. 2 also shows a first axis 202 defined by a first channel (e.g., well 112-1) and a second axis 204 defined by a second channel (e.g., well 112-2). In some embodiments, the first axis 202 is parallel to a longitudinal direction of the first channel (e.g., well 112-1). In some embodiments, the first axis 202 passes through a center of the first channel. In some embodiments, the second axis 204 is parallel to a longitudinal direction of the second channel (e.g., well 112-2). In some embodiments, the second axis 204 passes through a center of the second channel. In some embodiments, the first axis 202 is parallel to a height-wise direction of the first channel (e.g., well 112-1). In some embodiments, the second axis 204 is parallel to a height-wise direction of the second channel (e.g., well 112-2). In some embodiments, the first axis 202 is parallel to the second axis 204. In some embodiments, the first axis 202 is non-parallel to the second axis 204 (e.g., the first axis 202 is at a predefined non-zero angle from the second axis 204, such as 1 degree, 5 degrees, 10 degrees, 15 degrees, 20 degrees, or between any two angles selected from the aforementioned angles).

Although FIGS. 1 and 2 illustrate a plate with a plurality of wells, a receptacle having only a single well or channel (e.g., a tube) may be used instead, or in addition.

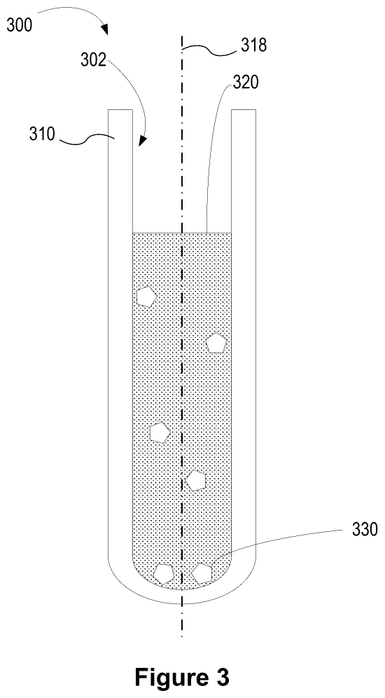

FIG. 3 illustrates a tube 300 in accordance with some embodiments. The tube 300 includes a tube wall 310, which defines a channel 302. The channel 302 has, or defines, an axis 318. In some embodiments, the axis 318 is parallel to the longitudinal direction of the channel 302.

FIG. 3 also illustrates that the tube 300 includes a solution 320 containing biological cells 330 or particles in some cases. However, as a person having ordinary skill in the art would recognize, the solution 320 and the biological cells 330 are not part of the tube 300.

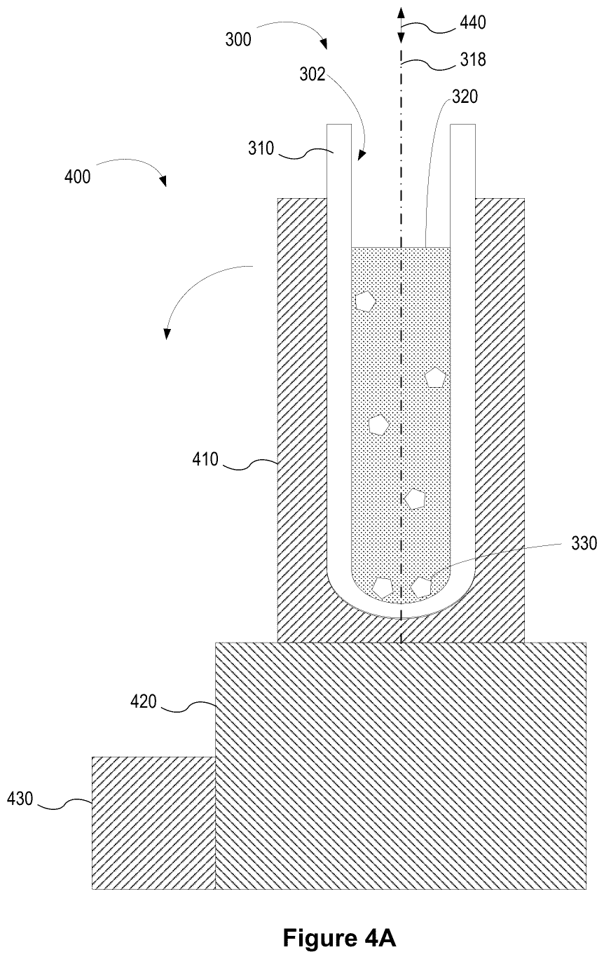

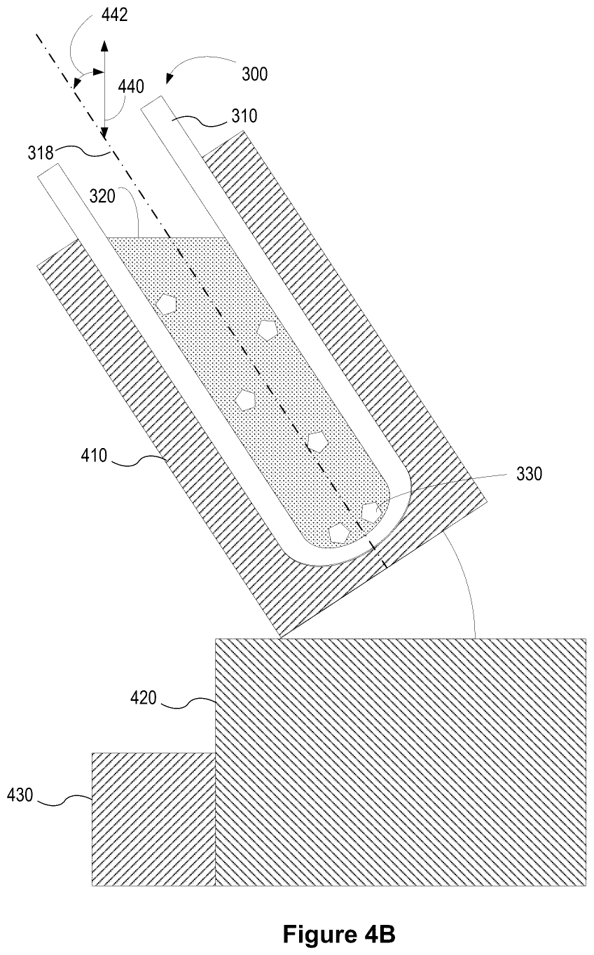

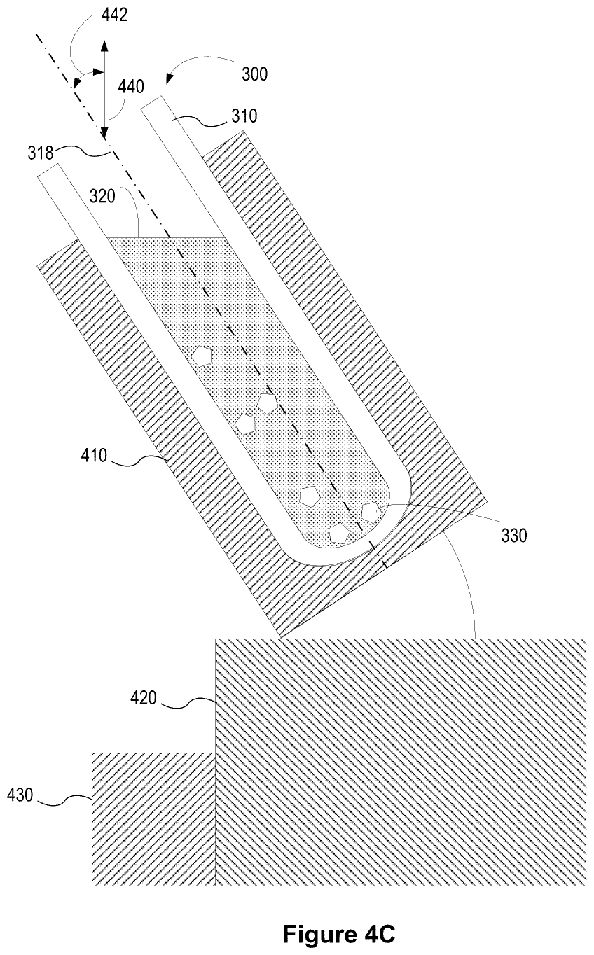

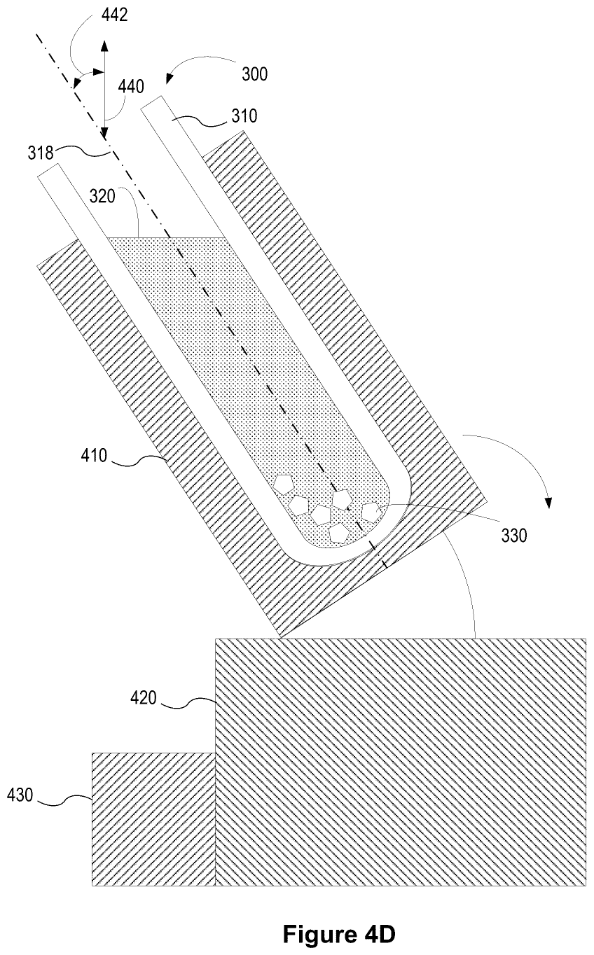

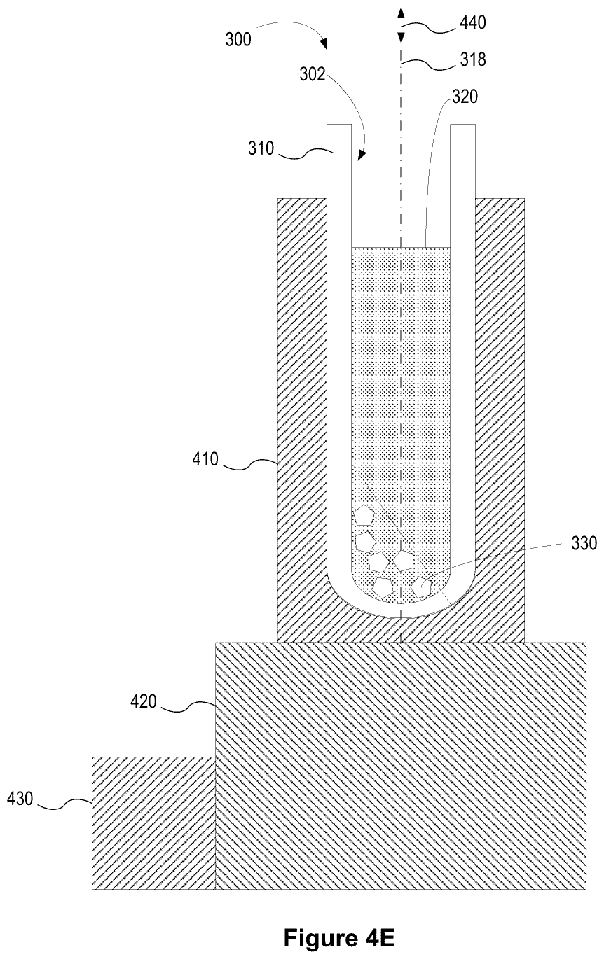

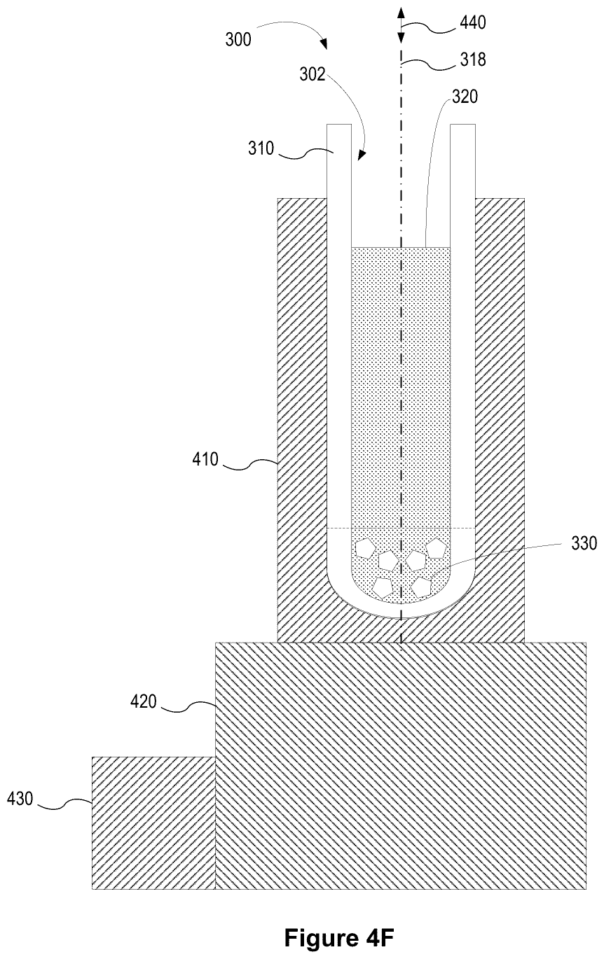

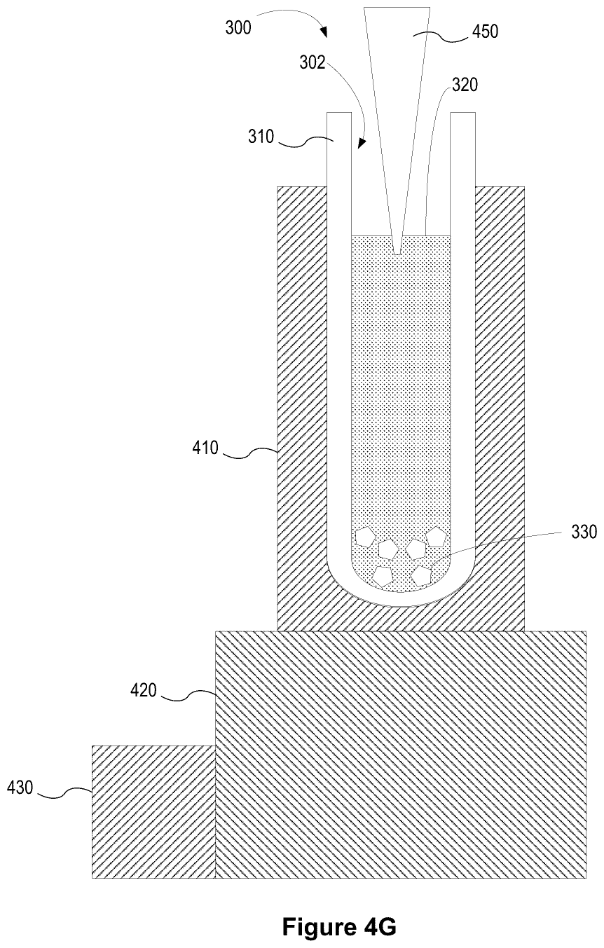

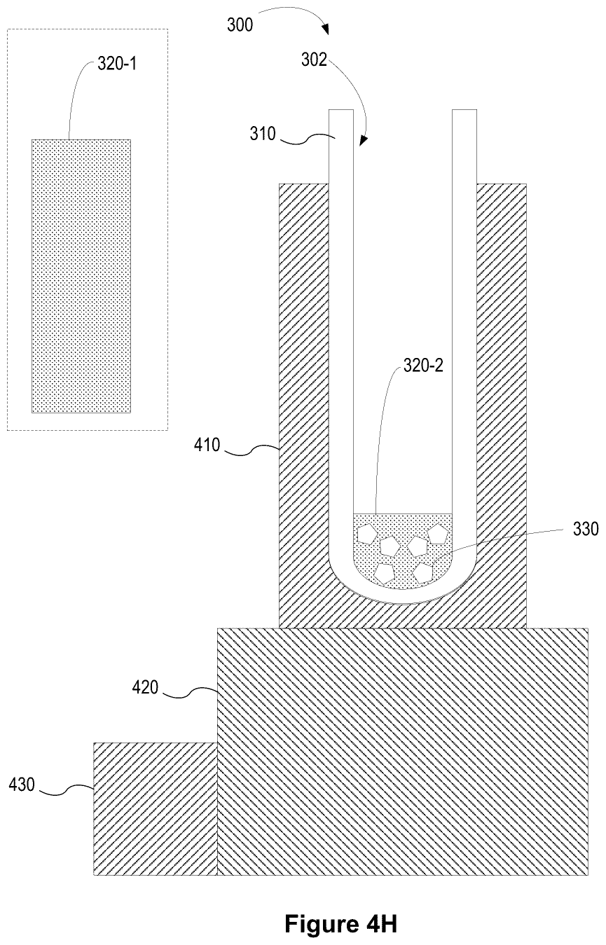

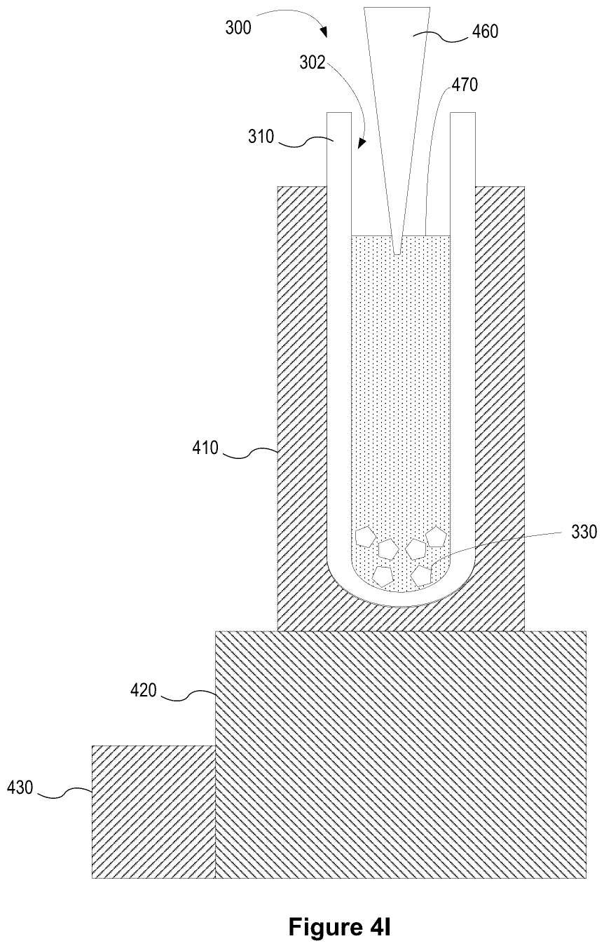

FIGS. 4A-4H illustrate an apparatus 400 for separating biological cells and operations for separating biological cells in accordance with some embodiments.

In FIG. 4A, the apparatus includes a receptacle holder 410 and a tilting device 420. In some embodiments, the apparatus also includes an impulse device 430.

As shown in FIG. 4A, in operation, a receptacle 300 (e.g., a tube) containing a solution 320 with biological cells 330 or particles may be placed in the receptacle holder 410.

In some embodiments, the receptacle holder 410 is in a second orientation. In some embodiments, the second orientation is a substantially vertical orientation as shown in FIG. 4A. In some embodiments, the receptacle holder 410 is deemed to be in a substantially vertical orientation when a channel defined by a receptacle to be held by the receptacle holder 410 defines an axis that is in a substantially vertical orientation. In some embodiments, the receptacle holder 410 (e.g., a tube holder) is deemed to be in a substantially vertical orientation when a channel defined by the receptacle holder 410 (e.g., a channel into which the tube is to be inserted) defines an axis that is in a substantially vertical orientation. For example, the first axis 318 defined by the channel 302 of the receptacle 300 or a channel of the receptacle holder 410 is substantially vertical (e.g., the axis 318 is substantially parallel to a vertical direction 440). In some embodiments, the receptacle holder 410 (e.g., a plate holder) is deemed to be in a substantially vertical orientation when a base surface of the receptacle holder 410 is substantially perpendicular to the vertical direction 440.

FIG. 4A also shows that the receptacle holder 410 holder is to be rotated or tilted by the tilting device 420.

FIG. 4B illustrates that the receptacle holder 410 is a first orientation. In some embodiments, the first orientation is a non-vertical orientation as shown in FIG. 4B. In some embodiments, the receptacle holder 410 is deemed to be in a non-vertical orientation when the channel defined by the receptacle to be held by the receptacle holder 410 defines an axis that is in a non-vertical orientation. In some embodiments, the receptacle holder 410 (e.g., a tube holder) is deemed to be in a non-vertical orientation when a channel defined by the receptacle holder 410 (e.g., a channel into which the tube is to be inserted) defines an axis that is in a non-vertical orientation (e.g., the axis has a non-zero angle with respect to the vertical direction). For example, the first axis 318 defined by the channel 302 of the receptacle 300 or a channel of the receptacle holder 410 is non-vertical (e.g., the axis 318 is non-parallel to the vertical direction 440). In some embodiments, the receptacle holder 410 (e.g., a plate holder) is deemed to be in a non-vertical orientation when a base surface of the receptacle holder 410 is non-perpendicular to the vertical direction 440.

FIG. 4C shows that the receptacle holder 410 is maintained in the first orientation. The tilting of the channel defined by the receptacle 310 speeds up settling of the biological cells or particles.

FIG. 4D shows that the receptable holder 410 continues to be maintained in the first orientation so that the tilting of the channel defined by the receptacle 310 continues to facilitate settling of the biological cells or particles.

FIG. 4D also shows that the receptacle holder 410 is to be rotated or tilted back by the tilting device 420.

FIG. 4E illustrates that the receptacle holder 410 is in the second orientation. In FIG. 4E, the biological cells 330 or particles are settled down to a bottom of the receptacle. In some cases, because the biological cells 330 or particles settled while the receptacle was in a tilted orientation, the biological cells 330 or particles may have settled in a non-symmetric manner (e.g., the biological cells 330 or particles may pile more on one end than an opposing end when the receptacle is positioned in a vertical orientation). For example, a top surface defined by the settled biological cells 330 or particles may be non-perpendicular to the vertical direction while the receptacle holder 410 or the receptacle 300 is in the vertical orientation as shown in FIG. 4E.

FIG. 4F illustrates that, in some embodiments, one or more mechanical impulses are provided to the receptacle 310 or the receptacle holder 410. In some cases, the one or more mechanical impulses cause the biological cells 330 or particles to redistribute. In some cases, the redistribution of the biological cells 330 or particles causes a top surface defined by the settled biological cells 330 or particles to be substantially perpendicular to the vertical direction while the receptacle holder 410 or the receptacle 300 is in the vertical orientation as shown in FIG. 4F.

FIG. 4G illustrates an aspirator 450 or its tip. In some embodiments, the aspirator 450 aspirates at least a portion of the solution 320 (and any substances not settled down with the biological cells 330 or particles, such as non-cellular substances).

FIG. 4H illustrates that a portion (e.g., a large portion) of the solution 320 is aspirated. A remaining portion 330-2 of the solution 320 with the biological cells 330 is left in the receptacle 300. The aspirated portion 320-1 is also illustrated in the inset of FIG. 4H to show that the aspirated portion 320-1 has no or a low concentration of biological cells whereas the remaining portion 330-2 has a high concentration of biological cells.

FIG. 4I illustrates a dispenser 460 or its tip. In some embodiments, the dispenser 460 is distinct from the aspirator 450. In some embodiments, the dispenser 460 dispenses a different solution (e.g., a wash buffer), which is mixed with the remaining portion of the solution 320 to form a mixed solution 470. By providing the different solution, the concentration of any substances not settled down with the biological cells 330 or particles, such as non-cellular substances, decreases.

In some embodiments, the aspiration of a portion of a solution in the receptacle and dispensing of additional solution are repeated to further decrease the concentration of any substances not settled down with the biological cells 330 or particles, such as non-cellular substances.

In some embodiments, the dispenser 460 or another dispenser provides one or more reagents. For example, in some embodiments, the dispenser 460 or another dispenser provides a lysis buffer. In some cases, provision of the lysis buffer causes lysis of at least certain biological cells (e.g., red blood cells) in the solution (e.g., whole blood). In some embodiments, the lysis buffer is provided at any stage described herein (e.g., before tilting).

Although the operations in FIGS. 4A-4I are illustrated with a tube, corresponding operations may be performed using a plate (e.g., the plate 100 shown in FIGS. 1 and 2). For brevity, such details are not repeated herein. The use of a plate having multiple channels allows concurrent separation operations for multiple samples or solutions.

### Example Whole Blood Washing Procedure

A whole blood sample was added to a receptacle (e.g., a well plate) and lysis buffer was added.

The receptacle was tilted and maintained in the tilted orientation for 35 minutes so that particles in a mixture of the whole blood and the lysis buffer settle.

After 35 minutes, a side of the plate was tapped (e.g., using a tapping block without a hammer head).

The receptacle was returned to an upright orientation (e.g., in case of a well plate, a horizontal orientation so that channels of the well plate are oriented upright)) and maintained in the upright orientation for 10 minutes so that the particles in the mixture can settle further.

A solution is aspirated from the receptacle (e.g., from one or more wells).

### Experimental Results

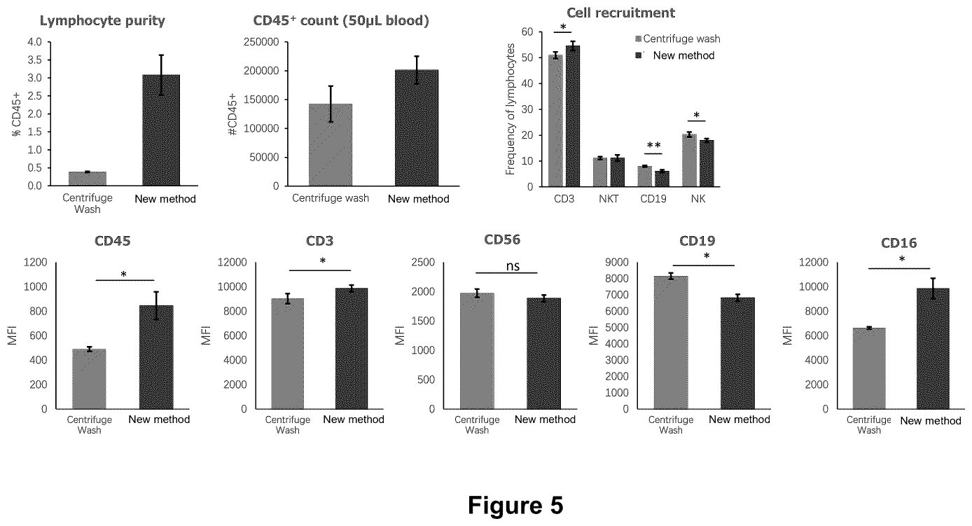

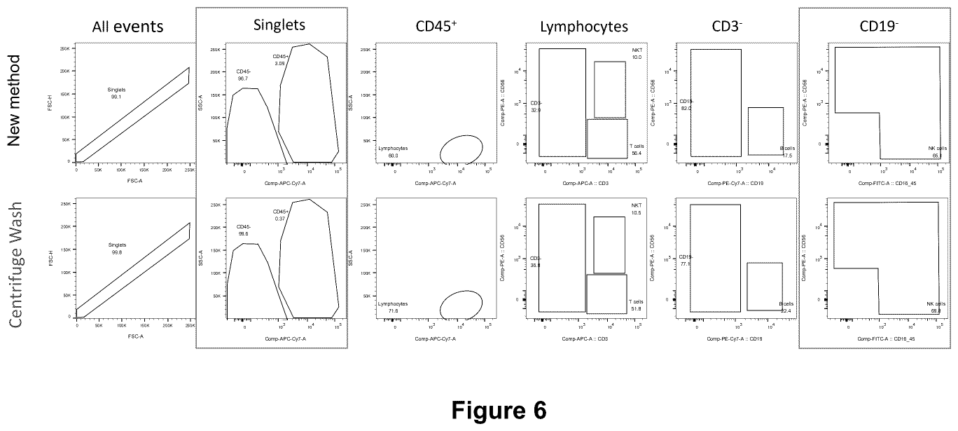

FIGS. 5 and 6 illustrate experimental results obtained by using the methods described herein.

FIG. 5 illustrates cell population frequencies and stain indices. Whole blood was stained with the TBNK reagent (BD Biosciences). The whole blood sample was split into two separate samples. One sample was washed using centrifugation (e.g., formation of the sediments by centrifugation, followed by aspiration of the solution and addition of a new buffer). The other sample was washed using the methods described herein, without using centrifugation. The results show that the cell population frequencies and stain indices are comparable between centrifuge-based washing and centrifuge-less washing.

FIG. 6 illustrates dot plots obtained by flow cytometry analysis of cells in whole blood stained with TBNK. The dot plots show that centrifuge-less washing described in this application provides clear resolution of neutrophils and natural killer (NK) cell population.

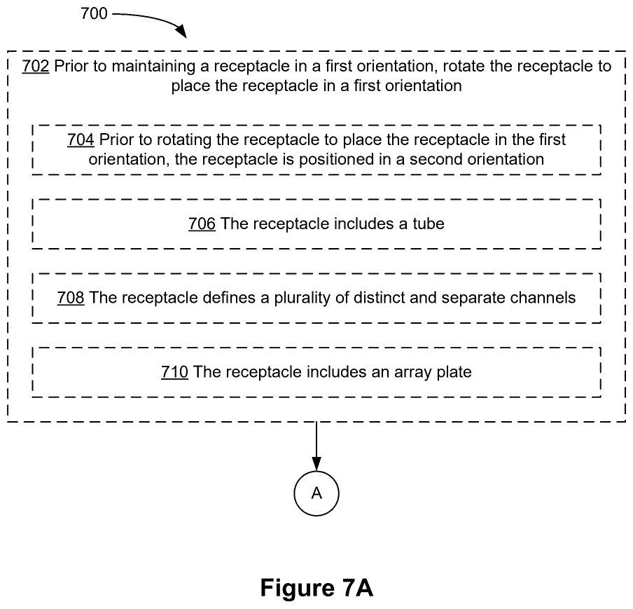

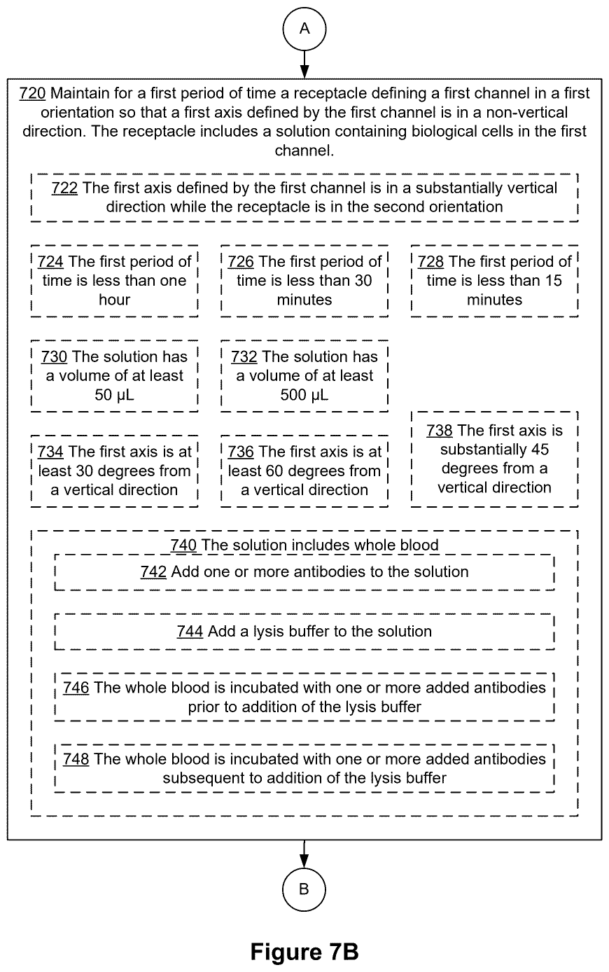

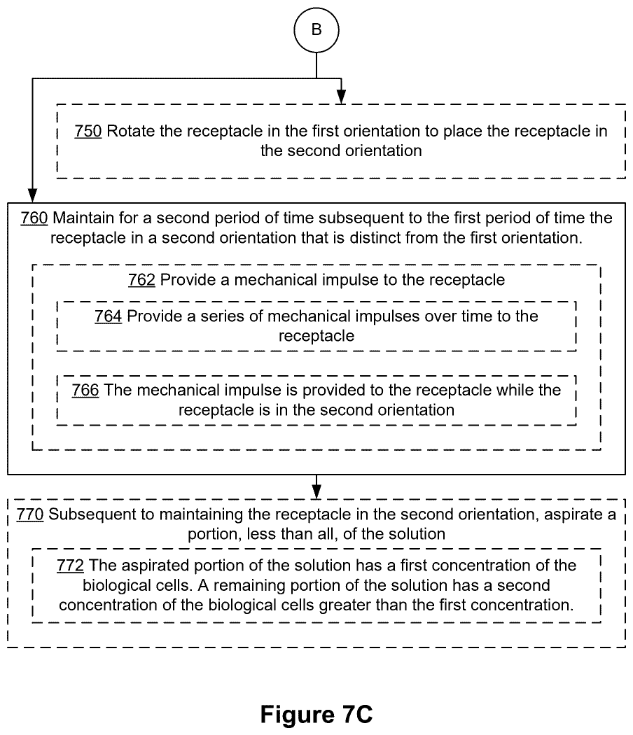

FIGS. 7A-7C are flow diagrams illustrating a method 700 of concentrating biological cells in accordance with some embodiments.

The method 700 includes (720) maintaining for a first period of time a receptacle (e.g., a tube 300 or a plate 100) defining a first channel (e.g., channel 302) in a first orientation (e.g., a non-vertical orientation as shown in FIG. 4C) so that a first axis (e.g., axis 318) defined by the first channel is in a non-vertical direction (e.g., the axis 318 in FIG. 4C). The receptacle includes a solution (e.g., solution 320) containing biological cells (e.g., cells 330) in the first channel.

In some embodiments, the method includes, prior to maintaining the receptacle in the first orientation, (702) rotating the receptacle to place the receptacle in the first orientation (e.g., rotation of the receptacle 310 from the vertical orientation shown in FIG. 4A to the non-vertical orientation shown in FIG. 4B).

In some embodiments, prior to rotating the receptacle to place the receptacle in the first orientation, the receptacle is positioned (704) in the second orientation (e.g., FIG. 4A).

In some embodiments, the receptacle includes (706) a tube (e.g., tube 300).

In some embodiments, the receptacle defines (708) a plurality of distinct and separate channels (e.g., the plate 100 with multiple wells, where each well corresponds to a respective channel).

In some embodiments, the receptacle includes (710) an array plate (e.g., the plate 100).

In some embodiments, the first axis defined by the first channel is (722) in a substantially vertical direction while the receptacle is in the second orientation (e.g., FIG. 4D).

In some embodiments, the first period of time is (724) less than one hour.

In some embodiments, the first period of time is (726) less than 30 minutes.

In some embodiments, the first period of time is (728) less than 15 minutes.

In some embodiments, the solution has (730) a volume of at least 50 μL. In some embodiments, the solution has (732) a volume of at least 500 μL.

In some embodiments, the first axis is (734) at least 30 degrees from a vertical direction. In some embodiments, the first axis is (736) substantially 45 degrees from a vertical direction. In some embodiments, the first axis is (738) at least 60 degrees from a vertical direction.

In some embodiments, the solution includes (740) whole blood.

In some embodiments, the method includes (742) adding one or more antibodies to the solution.

In some embodiments, the method includes adding (744) a lysis buffer to the solution. In some embodiments, the whole blood is incubated (746) with one or more added antibodies prior to addition of the lysis buffer. In some embodiments, the whole blood is incubated (748) with one or more added antibodies subsequent to addition of the lysis buffer.

In some embodiments, the method includes (750) rotating the receptacle in the first orientation to place the receptacle in the second orientation (e.g., rotating the receptacle 310 in the non-vertical orientation shown in FIG. 4C to the vertical orientation shown in FIG. 4D).

The method also includes (760) maintaining for a second period of time subsequent to the first period of time the receptacle in a second orientation (e.g., a vertical orientation as shown in FIG. 4D) that is distinct from the first orientation.

In some embodiments, the second period of time is less than 30 minutes, 20 minutes, 15 minutes, 10 minutes, 5 minutes, 3 minutes, 2 minutes, 1 minute, or within an interval between any two aforementioned values. In some embodiments, the second period of time is at least 60 seconds, 50 seconds, 40 seconds, 30 seconds, 20 seconds, 10 seconds, 5 seconds, 3 seconds, 2 seconds, 1 second, or within an interval between any two aforementioned values. In some embodiments, the second period of time is at least one second.

In some embodiments, the method includes (762) providing a mechanical impulse to the receptacle (e.g., tapping). In some embodiments, the mechanical impulse is provided directly to the receptacle. In some embodiments, the mechanical impulse is provided indirectly to the receptacle (e.g., by providing the mechanical impulse to the receptacle holder).

In some embodiments, the method includes (764) providing a series of mechanical impulses over time to the receptacle. For example, a sequence of multiple mechanical pulses may be provided over time.

In some embodiments, the mechanical impulse is provided (766) to the receptacle while the receptacle is in the second orientation (e.g., FIG. 4D).

In some embodiments, the method includes, subsequent to maintaining the receptacle in the second orientation, (770) aspirating a portion, less than all, of the solution (e.g., FIG. 4F).

In some embodiments, the aspirated portion (e.g., the aspirated portion 320-1) of the solution has (772) a first concentration of the biological cells; and a remaining portion of the solution (e.g., the remaining portion 320-2) has a second concentration of the biological cells greater than the first concentration. For example, the aspirated portion has a low concentration of the biological cells and the remaining portion of the solution has a high concentration of the biological cells, as illustrated in FIG. 4G.

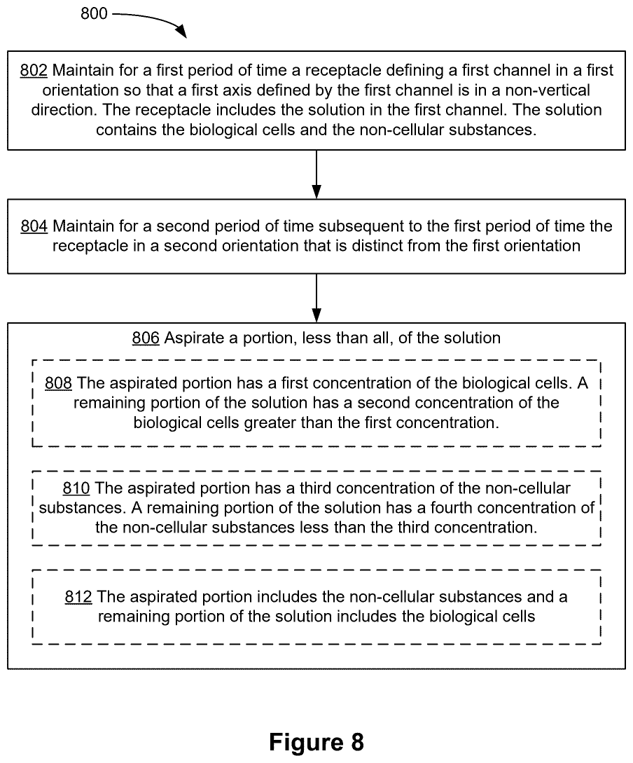

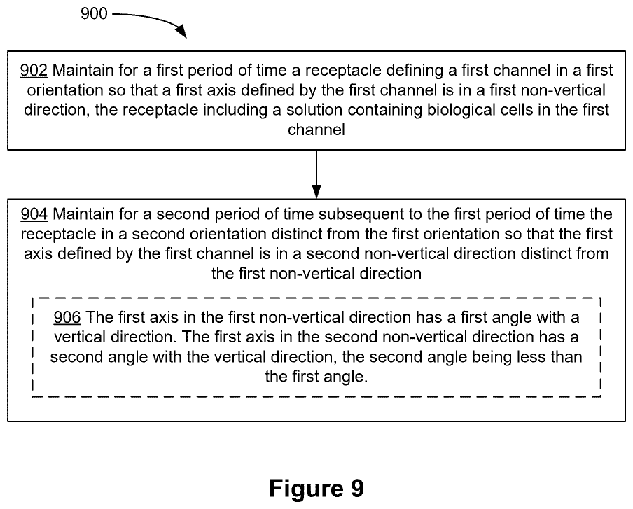

In some embodiments, some of the operations described with FIGS. 8 and 9 may be combined with the method 700. For brevity, such details are not repeated herein.

FIG. 8 is a flow diagram illustrating a method 800 of separating non-cellular substances from biological cells in accordance with some embodiments.

The method 800 for separating non-cellular substances from biological cells in a solution includes (802) maintaining for a first period of time a receptacle defining a first channel in a first orientation so that a first axis defined by the first channel is in a non-vertical direction. The receptacle includes the solution in the first channel, the solution containing the biological cells and the non-cellular substances.

The method also includes (804) maintaining for a second period of time subsequent to the first period of time the receptacle in a second orientation that is distinct from the first orientation.

The method further includes (806) aspirating a portion, less than all, of the solution.

In some embodiments, the aspirated portion has (808) a first concentration of the biological cells; and a remaining portion of the solution has a second concentration of the biological cells greater than the first concentration.

In some embodiments, the aspirated portion has (810) a third concentration of the non-cellular substances; and a remaining portion of the solution has a fourth concentration of the non-cellular substances less than the third concentration.

In some embodiments, the aspirated portion includes (812) the non-cellular substances and a remaining portion of the solution includes the biological cells.

In some embodiments, some of the operations described with FIGS. 7A-7C and 9 may be combined with the method 800. For brevity, such details are not repeated herein.

FIG. 9 is a flow diagram illustrating a method 900 of concentrating biological cells in a receptable defining a channel in accordance with some embodiments.

The method 900 includes (902) maintaining for a first period of time a receptacle defining a first channel in a first orientation so that a first axis defined by the first channel is in a first non-vertical direction. The receptacle includes a solution containing biological cells in the first channel.

The method also includes (904) maintaining for a second period of time subsequent to the first period of time the receptacle in a second orientation distinct from the first orientation so that the first axis defined by the first channel is in a second non-vertical direction distinct from the first non-vertical direction.

In some embodiments, the first axis in the first non-vertical direction has (906) a first angle with a vertical direction; and the first axis in the second non-vertical direction has a second angle with the vertical direction, the second angle being less than the first angle.

In some embodiments, some of the operations described with FIGS. 7A-7C and 8 may be combined with the method 900. For brevity, such details are not repeated herein.

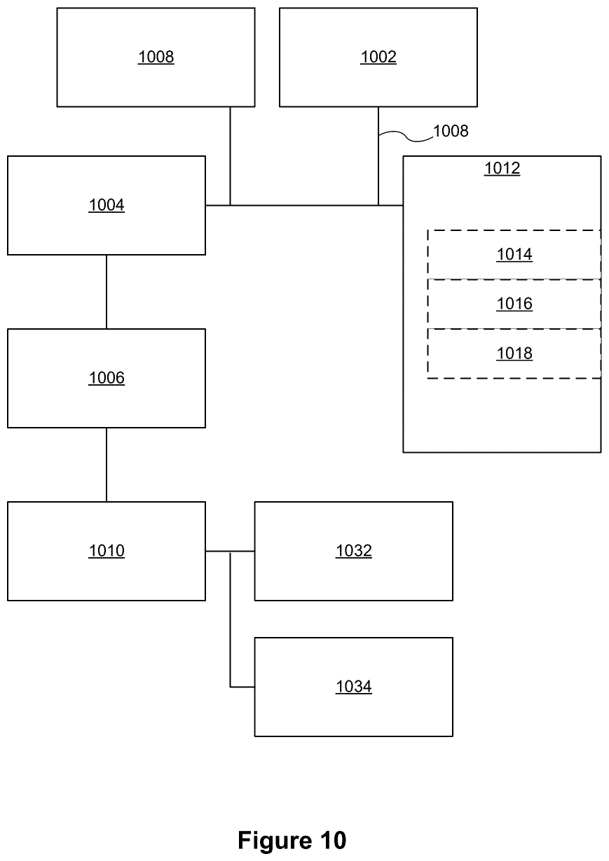

FIG. 10 is a block diagram illustrating electrical components of an apparatus in accordance with some embodiments.

The apparatus includes one or more one or more processors 1002 (central processing units, application processing units, application-specific integrated circuit, etc.), which are in communication (e.g., via one or more communication buses 1008 interconnecting a plurality of electronic components of the apparatus) with a computer readable storage medium 1012 (e.g., transitory computer readable storage medium or non-transitory computer memory devices, such as random-access memory, read-only memory, static random-access memory, and other non-volatile memory, and other storage devices, such as a hard drive, an optical disk, a magnetic tape recording, or any combination thereof) storing instructions for performing any methods described herein (e.g., operations described with respect to FIGS. 7A-7C, 8, and 9). For example, in some embodiments, the computer readable storage medium 1012 stores the following programs, modules, instructions, and data structures, or a subset thereof:
an operating system 1014 that includes procedures for handling various basic system services and for performing hardware dependent tasks;
a communication module (or instructions) 1016 that is used for connecting controller 1006 to other electronic devices via one or more communications interfaces 1004 and one or more communications networks (e.g., wired and/or wireless communication networks); and
actuator control instructions 1018 that causes electrical signals to be provided to one or

more actuators (directly from the one or more processors 1002 or through a driver 1010).

In some embodiments, the apparatus includes the one or more communication interfaces 1004 for communicating with other electronic devices.

In some embodiments, the controller 1006 includes, or is electrically coupled with, one or more drivers 1010 (via a system bus or any suitable electrical circuit). In some embodiments, the one or more drivers 1010 receives instructions and/or data from the one or more processors 1002 and relays the instructions and/or electrical signals to one or more actuators, such as a first actuator 1032 (e.g., in the tilting device 420), a second actuator 1034 (e.g., in the impulse device 430), etc.

In some embodiments, the apparatus includes, or is in communication with, one or more user interface (UI) devices 1008. In some embodiments, the UI devices 1008 include one or more user input devices (e.g., a keyboard, a mouse, a touch-sensitive surface, buttons, switches, etc.) for receiving user inputs and/or one or more output devices (e.g., a display, one or more indicators, an audio device, etc.) for providing an output to the user (e.g., a status of apparatus operations).

Although FIG. 10 shows that there is one processor 1002, in some embodiments, the apparatus includes additional processors. Similarly, although FIG. 10 shows one unit of computer readable storage medium 1012, in some embodiments, the computer readable storage medium 1012 is implemented across multiple physical devices. In some embodiments, the one or more processors 1002 are in communication with one or more user interface devices (e.g., displays and one or more user input devices, such as a keyboard, a mouse, a touch screen, etc.) for presenting information and/or receiving user inputs. Although FIG. 10 illustrates one or more communication interfaces 1004, controller 1006, and driver 1010 as separate devices, in some embodiments, at least some of one or more communication interfaces 1004, controller 1006, and driver 1010 may be combined or integrated.

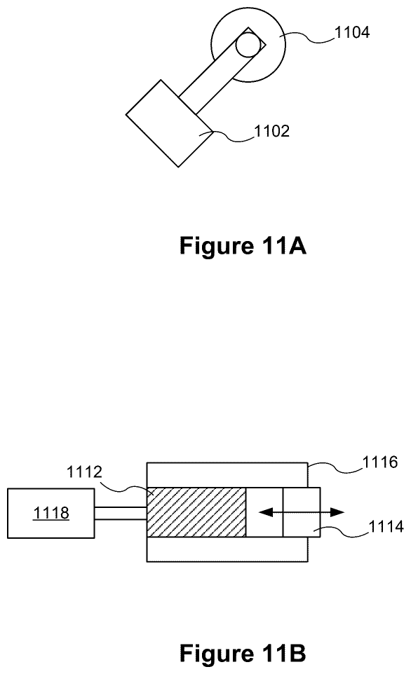

FIGS. 11A and 11B are schematic diagrams illustrating impulse devices in accordance with some embodiments.

FIG. 11A illustrates an impulse device that includes a hammer 1102. In FIG. 11A, the impulse device includes an actuator 1104 coupled with the hammer 1102. The actuator 1104 moves the hammer 1102 so that the hammer 1102 can provide a mechanical impulse (e.g., either by lifting and releasing the hammer 1102 so that the hammer 1102 drops and provides a mechanical impulse or by moving the hammer 1102 forward to provide a mechanical impulse). In some embodiments, the impulse device includes a tapping block (e.g., a block without a hammer head) for providing a mechanical impulse instead of the hammer 1102.

FIG. 11B illustrates an impulse device that includes an electromagnetic coil 1112 and an actuator 1114. In some embodiments, the actuator 1114 includes a magnet. The electromagnetic coil 1112 is electrically coupled with an electrical source 1118 (e.g., the driver 1010, the controller 1006, etc.). Based on the electrical signal from the electrical source 1118, the electromagnetic coil 1112 moves the actuator 1114 so that the impulse device provides a mechanical impulse.

In light of these principles and examples, we now turn to certain embodiments.

In accordance with some embodiments, an apparatus (e.g., the apparatus 400) includes a receptacle holder (e.g., the receptacle holder 410) for holding a receptacle, and a tilting device (e.g., the tilting device 420) coupled with the receptacle holder for placing the receptacle holder in a first orientation at a first time and placing the receptacle holder in a second orientation distinct from the first orientation at a second time distinct from the first time.

In some embodiments, the apparatus includes one or more processors (e.g., processor 1002) in communication with the tilting device (e.g., actuator 1032 in the tilting device); and memory (e.g., computer readable storage medium 1012) storing instructions for execution by the one or more processors. The stored instructions including instructions for:
sending a first set of one or more signals to the tilting device for placing the receptacle holder in the first orientation; and sending a second set of one or more signals to the tilting device for placing the receptable holder in the second orientation.

In some embodiments, the stored instructions include instructions for: sending the first set of one or more signals to the tilting device to the tilting device for placing the receptacle holder in the first orientation at a first time; and sending the second set of one or more signals to the tilting device for placing the receptacle holder in the second orientation at a second time subsequent to the first time so that the receptacle holder is maintained in the first orientation for a first period of time.

In some embodiments, the first period of time is less than one hour.

In some embodiments, the first period of time is less than 30 minutes.

In some embodiments, the first period of time is less than 15 minutes.

In some embodiments, the apparatus forgoes, subsequent to sending the second set of one or more signals, sending a set of one or more signals to the tilting device for placing the receptable holder in any orientation other than the second orientation for at least a second period of time from sending the second set of one or more signals.

In some embodiments, the stored instructions include instructions for: prior to sending the first set of one or more signals to the tilting device for placing the receptacle holder in the first orientation, sending one or more signals to the tilting device for placing the receptacle holder in the second orientation.

In some embodiments, the receptacle holder includes a tube rack (e.g., the receptacle holder 410).

In some embodiments, the receptacle includes a tube (e.g., the tube 300).

In some embodiments, the receptacle holder includes a holder for an array plate.

In some embodiments, the receptacle includes an array plate.

In some embodiments, the apparatus includes an aspirator (e.g., the aspirator 450) for aspirating a portion of a solution in a first channel defined in the receptacle.

In some embodiments, the apparatus includes a dispenser (e.g., the dispenser 460) for dispensing a wash buffer to a solution in a first channel defined in the receptacle.

In some embodiments, the apparatus includes a liquid transferrer (e.g., a pipette). The liquid transferrer is capable of both aspirating liquid and dispensing liquid.

In some embodiments, the apparatus includes one or more mechanical or electromechanical components (e.g., the impulse device 430 or one or more components thereof) for providing a mechanical impulse to the receptacle.

In some embodiments, the one or more mechanical or electromechanical components include a hammer (e.g., hammer 1102).

In some embodiments, the one or more mechanical or electromechanical components include an electromagnetic coil (e.g., coil 1112) and an actuator (e.g., actuator 1114).

In some embodiments, the receptacle defines a first channel along a first axis. While the receptacle holder is in the first orientation, the receptacle is in the first orientation; and the first axis is in a non-vertical direction.

In some embodiments, while the receptacle holder is in the second orientation, the receptacle is in the second orientation; and the first axis is in a substantially vertical direction.

In some embodiments, while the receptacle holder is in the first orientation, the first axis is at least 30 degrees from a vertical direction.

In some embodiments, the first axis is at least 30 degrees from a vertical direction.

In some embodiments, the first axis is substantially 45 degrees from the vertical direction.

In some embodiments, the first axis is at least 60 degrees from the vertical direction.

In some embodiments, the apparatus includes a dispenser for dispensing one or more antibodies.

In some embodiments, the apparatus includes a dispenser for dispensing a lysis buffer.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

Some of the embodiments are described with reference to the following clauses.
Clause 1. A method, comprising:
   maintaining for a first period of time a receptacle defining a first channel in a first orientation so that a first axis defined by the first channel is in a non-vertical direction, the receptacle including a solution containing biological cells in the first channel; and
maintaining for a second period of time subsequent to the first period of time the receptacle in a second orientation that is distinct from the first orientation.
Clause 2. The method of clause 1, wherein the first axis defined by the first channel is in a substantially vertical direction while the receptacle is in the second orientation.
Clause 3. The method of clause 1 or 2, further comprising: rotating the receptacle in the first orientation to place the receptacle in the second orientation.
Clause 4. The method of any of clauses 1-3, further comprising:
prior to maintaining the receptacle in the first orientation, rotating the receptacle to place the receptacle in the first orientation.
Clause 5. The method of clause 4, wherein:
   prior to rotating the receptacle to place the receptacle in the first orientation, the receptacle is positioned in the second orientation.
Clause 6. The method of any of clauses 1-5, wherein:
   the first period of time is less than one hour.
Clause 7. The method of clause 6, wherein:
   the first period of time is less than 30 minutes.
Clause 8. The method of clause 7, wherein:
   the first period of time is less than 15 minutes.
Clause 9. The method of any of clauses 1-8, wherein:
   the solution has a volume of at least 50 μL.
Clause 10. The method of clause 9, wherein:
   the solution has a volume of at least 500 μL.
Clause 11. The method of any of clauses 1-10, wherein:
   the first axis is at least 30 degrees from a vertical direction.
Clause 12. The method of any of clauses 1-11, wherein:
   the first axis is substantially 45 degrees from a vertical direction.
Clause 13. The method of any of clauses 1-11, wherein:
   the first axis is at least 60 degrees from a vertical direction.
Clause 14. The method of any of clauses 1-13, wherein:
   the solution includes whole blood.
Clause 15. The method of clause 14, further comprising:
   adding one or more antibodies to the solution.
Clause 16. The method of clause 14 or 15, further comprising:
   adding a lysis buffer to the solution.
Clause 17. The method of clause 16, wherein:
   the whole blood is incubated with one or more added antibodies prior to addition of the lysis buffer.
Clause 18. The method of clause 16, wherein:
   the whole blood is incubated with one or more added antibodies subsequent to addition of the lysis buffer.
Clause 19. The method of any of clauses 1-18, further comprising:
   subsequent to maintaining the receptacle in the second orientation, aspirating a portion, less than all, of the solution.
Clause 20. The method of clause 19, wherein:
   the aspirated portion of the solution has a first concentration of the biological cells; and
   a remaining portion of the solution has a second concentration of the biological cells greater than the first concentration.
Clause 21. The method of any of clauses 1-19, wherein:
   the receptacle includes a tube.
Clause 22. The method of any of clauses 1-19, wherein:
   the receptacle defines a plurality of distinct and separate channels.
Clause 23. The method of any of clauses 1-20 and 22, wherein:
   the receptacle includes an array plate.
Clause 24. The method of any of clauses 1-23, further comprising:
   providing a mechanical impulse to the receptacle.
Clause 25. The method of clause 24, further comprising:
   providing a series of mechanical impulses over time to the receptacle.
Clause 26. The method of clause 24 or 25, wherein:
   the mechanical impulse is provided to the receptacle while the receptacle is in the second orientation.
Clause 27. An apparatus, comprising:
   a receptacle holder for holding a receptacle; and
   a tilting device coupled with the receptacle holder for placing the receptacle holder in a first orientation at a first time and placing the receptacle holder in a second orientation distinct from the first orientation at a second time distinct from the first time.
Clause 28. The apparatus of clause 27, further comprising:
   one or more processors in communication with the tilting device; and
   memory storing instructions for execution by the one or more processors, the stored instructions including instructions for:
sending a first set of one or more signals to the tilting device for placing the receptacle holder in the first orientation; and
sending a second set of one or more signals to the tilting device for placing the receptable holder in the second orientation.
Clause 29. The apparatus of clause 28, wherein:
   the stored instructions include instructions for:
sending the first set of one or more signals to the tilting device to the tilting device for placing the receptacle holder in the first orientation at a first time; and
sending the second set of one or more signals to the tilting device for placing the receptacle holder in the second orientation at a second time subsequent to the first time so that the receptacle holder is maintained in the first orientation for a first period of time.
Clause 30. The apparatus of clause 29, wherein:
   the first period of time is less than one hour.
Clause 31. The apparatus of clause 30, wherein:
   the first period of time is less than 30 minutes.
Clause 32. The apparatus of clause 31, wherein:
   the first period of time is less than 15 minutes.
Clause 33. The apparatus of any of clauses 28-32, wherein:
The apparatus forgoes, subsequent to sending the second set of one or more signals, sending a set of one or more signals to the tilting device for placing the receptable holder in any orientation other than the second orientation for at least a second period of time from sending the second set of one or more signals.

Clause 34. The apparatus of any of clauses 28-33, wherein:

the stored instructions include instructions for:
prior to sending the first set of one or more signals to the tilting device for placing the receptacle holder in the first orientation, sending one or more signals to the tilting device for placing the receptacle holder in the second orientation.
Clause 35. The apparatus of any of clauses 27-34, wherein:
   the receptacle holder includes a tube rack.
Clause 36. The apparatus of any of clauses 27-35, wherein:
   the receptacle includes a tube.
Clause 37. The apparatus of any of clauses 27-36, wherein:
   the receptacle holder includes a holder for an array plate.
Clause 38. The apparatus of any of clauses 27-37, wherein:
   the receptacle includes an array plate.
Clause 39. The apparatus of any of clauses 27-38, further comprising:
   an aspirator for aspirating a portion of a solution in a first channel defined in the receptacle.
Clause 40. The apparatus of any of clauses 27-39, further comprising:
   a dispenser for dispensing a wash buffer to a solution in a first channel defined in the receptacle.
Clause 41. The apparatus of any of clauses 27-40, further comprising:
   one or more mechanical or electromechanical components for providing a mechanical impulse to the receptacle.
Clause 42. The apparatus of clause 41, wherein:
   the one or more mechanical or electromechanical components include a hammer.
Clause 43. The apparatus of clause 41 or 42, wherein:
   the one or more mechanical or electromechanical components include an electromagnetic coil and an actuator.
Clause 44. The apparatus of any of clauses 27-43, wherein:
   the receptacle defines a first channel along a first axis;
   while the receptacle holder is in the first orientation:
the receptacle is in the first orientation; and
the first axis is in a non-vertical direction.
Clause 45. The apparatus of clause 44, wherein:
   while the receptacle holder is in the second orientation:
the receptacle is in the second orientation; and
the first axis is in a substantially vertical direction.
Clause 46. The apparatus of clause 44 or 45, wherein:
   while the receptacle holder is in the first orientation, the first axis is at least 30 degrees from a vertical direction.
Clause 47. The apparatus of clause 46, wherein:
   the first axis is at least 30 degrees from a vertical direction.
Clause 48. The apparatus of clause 47, wherein:
   the first axis is substantially 45 degrees from the vertical direction.
Clause 49. The apparatus of clause 47, wherein:
   the first axis is at least 60 degrees from the vertical direction.
Clause 50. The apparatus of any of clauses 27-49, further comprising:
   a dispenser for dispensing one or more antibodies.
Clause 51. The apparatus of any of clauses 27-50, further comprising:
   a dispenser for dispensing a lysis buffer.
Clause 52. A method for separating non-cellular substances from biological cells in a solution, the method comprising:
   maintaining for a first period of time a receptacle defining a first channel in a first orientation so that a first axis defined by the first channel is in a non-vertical direction, the receptacle including the solution in the first channel, the solution containing the biological cells and the non-cellular substances;
   maintaining for a second period of time subsequent to the first period of time the receptacle in a second orientation that is distinct from the first orientation; and
   aspirating a portion, less than all, of the solution.
Clause 53. The method of clause 52, wherein:
   the aspirated portion has a first concentration of the biological cells; and
   a remaining portion of the solution has a second concentration of the biological cells greater than the first concentration.
Clause 54. The method of clause 52 or 53, wherein:
   the aspirated portion has a third concentration of the non-cellular substances; and
   a remaining portion of the solution has a fourth concentration of the non-cellular substances less than the third concentration.
Clause 55. The method of any of clauses 52-54, wherein:
   the aspirated portion includes the non-cellular substances and a remaining portion of the solution includes the biological cells.
Clause 56. A method, comprising:
   maintaining for a first period of time a receptacle defining a first channel in a first orientation so that a first axis defined by the first channel is in a first non-vertical direction, the receptacle including a solution containing biological cells in the first channel; and
   maintaining for a second period of time subsequent to the first period of time the receptacle in a second orientation distinct from the first orientation so that the first axis defined by the first channel is in a second non-vertical direction distinct from the first non-vertical direction.
Clause 57. The method of clause 56, wherein:
   the first axis in the first non-vertical direction has a first angle with a vertical direction; and
   the first axis in the second non-vertical direction has a second angle with the vertical direction, the second angle being less than the first angle.

What is claimed is:

1. A method, comprising:
maintaining for a first period of time a receptacle defining a first channel in a first orientation so that a first axis defined by the first channel is in a non-vertical direction, the receptacle including a solution containing biological cells in the first channel; and
maintaining for a second period of time subsequent to the first period of time the receptacle in a second orientation that is distinct from the first orientation.

2. The method of claim 1, wherein the first axis defined by the first channel is in a substantially vertical direction while the receptacle is in the second orientation.

3. The method of claim 1, further comprising:
rotating the receptacle in the first orientation to place the receptacle in the second orientation.

4. The method of claim 1, further comprising:
prior to maintaining the receptacle in the first orientation, rotating the receptacle to place the receptacle in the first orientation.

5. The method of claim 4, wherein:
prior to rotating the receptacle to place the receptacle in the first orientation, the receptacle is positioned in the second orientation.

6. The method of claim 1, wherein:
the solution includes whole blood.

7. The method of claim 6, further comprising:
adding one or more antibodies to the solution.

8. The method of claim 6, further comprising:
adding a lysis buffer to the solution.

9. The method of claim 8, wherein:
the whole blood is incubated with one or more added antibodies subsequent to addition of the lysis buffer.

10. The method of claim 1, further comprising:
subsequent to maintaining the receptacle in the second orientation, aspirating a portion, less than all, of the solution.

11. The method of claim 10, wherein:
the aspirated portion of the solution has a first concentration of the biological cells; and
a remaining portion of the solution has a second concentration of the biological cells greater than the first concentration.

12. The method of claim 1, further comprising:
providing a mechanical impulse to the receptacle.

13. The method of claim 12, further comprising:
providing a series of mechanical impulses over time to the receptacle.

14. The method of claim 12, wherein:
the mechanical impulse is provided to the receptacle while the receptacle is in the second orientation.

15. A method for separating non-cellular substances from biological cells in a solution, the method comprising:
maintaining for a first period of time a receptacle defining a first channel in a first orientation so that a first axis defined by the first channel is in a non-vertical direction, the receptacle including the solution in the first channel, the solution containing the biological cells and the non-cellular substances;
maintaining for a second period of time subsequent to the first period of time the receptacle in a second orientation that is distinct from the first orientation; and
aspirating a portion, less than all, of the solution.

16. The method of claim 15, wherein:
the aspirated portion has a first concentration of the biological cells; and
a remaining portion of the solution has a second concentration of the biological cells greater than the first concentration.

17. The method of claim 15, wherein:
the aspirated portion has a third concentration of the non-cellular substances; and
a remaining portion of the solution has a fourth concentration of the non-cellular substances less than the third concentration.

18. The method of claim 15, wherein:
the aspirated portion includes the non-cellular substances and a remaining portion of the solution includes the biological cells.

19. A method, comprising:
maintaining for a first period of time a receptacle defining a first channel in a first orientation so that a first axis defined by the first channel is in a first non-vertical direction, the receptacle including a solution containing biological cells in the first channel; and
maintaining for a second period of time subsequent to the first period of time the receptacle in a second orientation distinct from the first orientation so that the first axis defined by the first channel is in a second non-vertical direction distinct from the first non-vertical direction.

20. The method of claim 19, wherein:
the first axis in the first non-vertical direction has a first angle with a vertical direction; and
the first axis in the second non-vertical direction has a second angle with the vertical direction, the second angle being less than the first angle.
